(19) United States
(12) Patent Application Publication
(10) Pub. No.: US 2010/0114110 A1
(43) Pub. Date: May 6, 2010

(54) INTERVERTEBRAL DISC ACCESS ASSEMBLY

(75) Inventors: Richard J. Taft, Austin, TX (US); Robert Nucci, Tampa, FL (US); George Morrison, Austin, TX (US); David Cox, Austin, TX (US)

Correspondence Address: ARTHROCARE CORPORATION;ATTN: Matthew Scheele, 7500 Rialto Boulevard, Building Two, Suite 100, Austin, TX, 78735-8532, US

(73) Assignee: ArthroCare Corporation, Austin, TX (US)

(21) Appl. No.: 12/261,142

(22) Filed: Oct. 30, 2008

(51) Int. Cl. A61F 11/00; A61B 18/14; A61M 5/24

(52) U.S. Cl. 606/108; 600/114; 600/201

(57) ABSTRACT

A surgical access system for managing the minimally invasive access of treatment electrodes to an intervertebral disc, when treating spine abnormalities such as disc herniations. The system includes an access port and a cannula assembly. The access port provides atraumatic access to the target area for the cannula system and a subsequent treatment electrode. The access port also manages the insertion travel and penetration depth of the cannula assembly, so as to minimize unintended damage to local tissue during use. The cannula system includes an adjustable and removable stop to mate with the access port that limits the extension of the cannula beyond the distal tip of the access port and into the target tissue.

### FIELD OF THE INVENTION

The present invention relates generally to an apparatus for accessing spinal discs or tissue structures and more particularly to an access assembly that incorporates an access port adapted to facilitate the protected and precise placement of a cannula assembly and treatment electrode within an intervertebral disc.

### BACKGROUND OF THE INVENTION

Intervertebral discs function to cushion and tether the vertebrae, while the interspinous tissue (i.e., tendons and cartilage, and the like) generally function to support the vertebrae so as to provide flexibility and stability to the patient's spine. Spinal discs comprise a central hydrophilic cushion, the nucleus pulposus, surrounded by a multi-layered fibrous ligament, the annulus fibrosus. As discs degenerate, they lose their water content and height, bringing the adjoining vertebrae closer together. This results in a weakening of the shock absorption properties of the disc and a narrowing of the nerve openings in the sides of the spine which may pinch these nerves. A weakening of the annulus fibrosus may cause the disc to bulge, e.g., a contained herniation, and the mere proximity of the nucleus pulposus or the damaged annulus to a nerve may cause direct pressure against the nerve, often resulting in persistent and debilitating pain as well as sensory and motor deficit.

Until recently, surgical spinal procedures typically included traumatic dissection of muscle, bone removal and/or bone fusion. To overcome the disadvantages of traditional traumatic spine surgery, less invasive techniques for spine surgery have been developed. Such minimally invasive techniques for the treatment of spinal diseases or disorders include chemonucleolysis, laser techniques, and mechanical techniques. These procedures generally require a surgeon to form a passage or operating corridor from the external surface of the patient to the spinal disc(s) for passage of surgical instruments, implants and the like. Typically, the formation of this operating corridor requires the damage or removal of soft tissue, muscle or other types of tissue depending on the procedure (i.e., laparascopic, thoracoscopic, arthroscopic, back, etc.). Recently, as a solution to many of the concerns with traditional mechanical instruments, energy based instrumentation, including radio frequency products, have been adapted for the treatment of intervertebral discs. Such energy based devices offer a wide range of treatment options such as ablation, shrinking, cauterization, and removing or dissolving target tissue within a disc or in and around the spine.

The area around the cervical spine presents additional challenges to performing minimally invasive procedures. Access to a diseased or degenerated cervical disc is frequently gained through the neck, in close vicinity to vital structures such as the carotid artery, trachea, deep veins of the neck and esophagus. Furthermore, access is frequently gained from an anterior or anterolateral approach, such that the surgeon is required to create an operating corridor while navigating around most of these vital structures. This operating corridor is then used to place and remove multiple devices such as small piercing access stylets, cutting mechanical instruments and energy based electrodes, any one of which may inadvertently damage a structure within the neck.

Additionally, the surgeon may also need to release hold of the inserted instruments during their positioning to ensure that the location or trajectory of the instruments is accurate. This information is usually obtained through radiographic visualization means such as fluoroscopic imaging or Computed Tomography (CT). There is a concern that while not holding an instrument in place that an instrument may unintentionally move or slip.

During cervical disc procedures there are generally two access approaches or routes that may be chosen: an anterior access route beginning at a point medial to the patient's neck and an anterolateral access route which begins at a point located more laterally. In procedures utilizing the anterolateral access approach, the patient will often lie with his or her head facing to the side. Which access point is chosen depends on the patient, surgeon preference, surgical protocol and where the disc degeneration has occurred. Additionally, the choice in access approach (e.g. anterior or anterolateral) effects the range of penetration depth available for treating the target tissue.

### SUMMARY OF THE INVENTION

Therefore a need has arisen for an improved system to provide a protected and controlled method for accessing and treating spinal discs including cervical discs.

A further need has arisen for an improved system for selectively predetermining and limiting the penetration depth of treatment instruments.

The present disclosure presents an improved surgical device system for managing the minimally invasive access of instruments to an intervertebral disc in the spine. The device system includes an access port and a cannula assembly which is removably inserted into the access port. The access port provides substantially atraumatic access to the target area for the cannula assembly and subsequent instrumentation. The cannula assembly includes at least one cannula and at least one removable stop. The access port also provides a method of managing the insertion depth of the cannula assembly so as to minimize unintended damage to surrounding patient tissue particularly the tissue beyond the target tissue. The removable stop may interface with the access port when assembled and provides a selectable hard-stop when the cannula is inserted into the access port. The adjustable stop advantageously limits the cannula extension beyond the access port tip and thereby limits the extension of the cannula and subsequent instrumentation into the target tissue.

In one aspect an access port is disclosed for providing access for a cannula assembly and treatment electrode to a target tissue. The access port includes a port handle and a smooth and rounded atraumatic port tubular elongate body extending from the port handle. The tubular elongate body has a proximal end and an inner luminal surface and this inner luminal surface also has at least one elastomeric frictional component that creates a frictional grip with any instrument or assembly that is inserted into the access port. This allows the user to let go of the advancing assembly or instrument without the likelihood that the assembly or instrument will move or slip to an unintended location. The proximal end of the tubular body also has an interface surface or nest, formed to interface with a stop that limits the insertion depth of the cannula assembly.

In another aspect, a surgical device system includes an access port and a retractor. This retractor aids in retracting the tissue away from the access port while minimizing any tissue damage. The retractor has a handle and a retraction portion connected with the handle, and the retraction portion may have a curved surface designed to nest with the access port to help guide the access port towards the target tissue.

In yet another aspect a method of performing a medical procedure on a body is disclosed. The method includes providing an access port for accessing a target tissue. The access port has a port handle and a port tubular elongate body extending from the port handle and the tubular elongate body has a proximal end and a distal end. The method also includes inserting the access port tubular body into a patient towards target tissue and then advancing the access port tubular body further to access the target tissue. The method further includes providing a cannula assembly. The cannula assembly includes a cannula, a stylet and a stop. The stop may be attached to a desired position on the cannula outer surface. The method then further includes inserting the cannula assembly into the access port tubular body until the stop mates with the proximal end of the port tubular body.

The present disclosure includes a number of important technical advantages. One technical advantage is that the access port is adapted to provide atraumatic access to the target tissue through the access port's smooth and rounded surfaces. In addition the port tube provides a protected conduit for potentially traumatic access and treatment assemblies. Another important technical advantage is that the cannula assembly stop provides an insertion depth limit for any advancing cannula assembly and subsequent instrumentation. This advantageously reduces the likelihood of over-insertion and any unintended tissue damage. Another advantage is that this stop may be varied in size and location to adapt to different patients and access points. Another important technical advantage comes from the access port frictional components, which allow the surgeon to release a partially inserted cannula assembly (inserted within the port) without the assembly slipping or moving out of position. Another important advantage is that the frictional components and port tube conduit provide an improved and accurate trajectory, allowing for a more precise approach for any inserted instrumentation. Additional advantages will be apparent to those of skill in the art and from the figures, description and claims provided herein.

### BRIEF DESCRIPTION OF THE DRAWINGS

FIG. 1 shows a surgical device system including an access port and cannula assembly according to the teachings of the present disclosure;

FIG. 2 shows cut-away view of an access port, having elastomeric frictional components and grooves according to the teachings of the present disclosure;

FIG. 3A shows an isometric view of a cannula assembly with a removable adjustable stop according to the teachings of the present disclosure;

FIG. 3B shows a cross section of a portion of a cannula assembly;

FIG. 4A shows a retractor and 4B shows a view of the retractor's distal tip;

FIG. 5 shows an access port, retractor, cannula, stop and treatment electrode assembled according to the teachings of the present disclosure;

FIG. 6 shows a treatment electrode;

FIG. 7 is a representation of an access assembly in relation to a patient according to teachings of the present disclosure;

FIG. 8 shows an access assembly with anterior access to a cervical disc;

FIG. 9 shows a access assembly with anterolateral access to a cervical disc; and

FIG. 10 shows a flow diagram of a method of performing a medical procedure according to teachings of the present disclosure.

### DETAILED DESCRIPTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

The systems of the present invention may be configured to address any application wherein an access system is needed to minimally invasively access a target site in order to perform a medical procedure. The subject systems are particularly suitable for treating all types of cervical intervertebral discs. In certain embodiments, the system further includes a treatment device suitably configured for treating the degenerative intervertebral disc.

The treatment device (which may also be referred to as a “treatment electrode” herein) of the present invention may have a variety of mechanical and/or electrosurgical configurations. However, one variation of the invention employs a treatment device using Coblation® technology.

As stated above, the assignee of the present invention developed Coblation® technology. Coblation® technology involves the application of a high frequency voltage difference between one or more active electrode(s) and one or more return electrode(s) to develop high electric field intensities in the vicinity of the target tissue. The high electric field intensities may be generated by applying a high frequency voltage that is sufficient to vaporize an electrically conductive fluid over at least a portion of the active electrode(s) in the region between the tip of the active electrode(s) and the target tissue. The electrically conductive fluid may be a liquid or gas, such as isotonic saline, blood, extracelluar or intracellular fluid, delivered to, or already present at, the target site, or a viscous fluid, such as a gel, applied to the target site.

When the conductive fluid is heated enough such that atoms vaporize off the surface faster than they recondense, a gas is formed. When the gas is sufficiently heated such that the atoms collide with each other causing a release of electrons in the process, an ionized gas or plasma is formed (the so-called “fourth state of matter”). Generally speaking, plasmas may be formed by heating a gas and ionizing the gas by driving an electric current through it, or by shining radio waves into the gas. These methods of plasma formation give energy to free electrons in the plasma directly, and then electron-atom collisions liberate more electrons and the process cascades until the desired degree of ionization is achieved. A more complete description of plasma can be found in Plasma Physics, by R. J. Goldston and P. H. Rutherford of the Plasma Physics Laboratory of Princeton University (1995), the complete disclosure of which is incorporated herein by reference.

As the density of the plasma or vapor layer becomes sufficiently low (i.e., less than approximately 1020 atoms/cm3 for aqueous solutions), the electron mean free path increases to enable subsequently injected electrons to cause impact ionization within the vapor layer. Once the ionic particles in the plasma layer have sufficient energy, they accelerate towards the target tissue. Energy evolved by the energetic electrons (e.g., 3.5 eV to 5 eV) can subsequently bombard a molecule and break its bonds, dissociating a molecule into free radicals, which then combine into final gaseous or liquid species. Often, the electrons carry the electrical current or absorb the radio waves and, therefore, are hotter than the ions. Thus, the electrons, which are carried away from the tissue towards the return electrode, carry most of the plasma's heat with them, allowing the ions to break apart the tissue molecules in a substantially non-thermal manner.

By means of this molecular dissociation (rather than thermal evaporation or carbonization), the target tissue structure is volumetrically removed through molecular disintegration of larger organic molecules into smaller molecules and/or atoms, such as hydrogen, oxygen, oxides of carbon, hydrocarbons and nitrogen compounds. This molecular disintegration completely removes the tissue structure, as opposed to dehydrating the tissue material by the removal of liquid within the cells of the tissue and extracellular fluids, as is typically the case with electrosurgical desiccation and vaporization. A more detailed description of this phenomenon can be found in commonly assigned U.S. Pat. No. 5,697,882 the complete disclosure of which is incorporated herein by reference.

In some applications of the Coblation® technology, high frequency (RF) electrical energy is applied in an electrically conducting media environment to shrink or remove (i.e., resect, cut, or ablate) a tissue structure and to seal transected vessels within the region of the target tissue. Coblation® technology is also useful for sealing larger arterial vessels, e.g., on the order of about 1 mm in diameter. In such applications, a high frequency power supply is provided having an ablation mode, wherein a first voltage is applied to an active electrode sufficient to effect molecular dissociation or disintegration of the tissue, and a coagulation mode, wherein a second, lower voltage is applied to an active electrode (either the same or a different electrode) sufficient to heat, shrink, and/or achieve hemostasis of severed vessels within the tissue.

The amount of energy produced by the Coblation® device may be varied by adjusting a variety of factors, such as: the number of active electrodes; electrode size and spacing; electrode surface area; asperities and sharp edges on the electrode surfaces; electrode materials; applied voltage and power; current limiting means, such as inductors; electrical conductivity of the fluid in contact with the electrodes; density of the fluid; and other factors. Accordingly, these factors can be manipulated to control the energy level of the excited electrons. Since different tissue structures have different molecular bonds, the Coblation® device may be configured to produce energy sufficient to break the molecular bonds of certain tissue but insufficient to break the molecular bonds of other tissue. For example, fatty tissue (e.g., adipose) has double bonds that require an energy level substantially higher than 4 eV to 5 eV (typically on the order of about 8 eV) to break. Accordingly, the Coblation® technology generally does not ablate or remove such fatty tissue; however, it may be used to effectively ablate cells to release the inner fat content in a liquid form. Of course, factors may be changed such that these double bonds can also be broken in a similar fashion as the single bonds (e.g., increasing voltage or changing the electrode configuration to increase the current density at the electrode tips). A more complete description of this phenomenon can be found in commonly assigned U.S. Pat. Nos. 6,355,032, 6,149,120 and 6,296,136, the complete disclosures of which are incorporated herein by reference.

The active electrode(s) of a Coblation® device may be supported within or by an inorganic insulating support positioned near the distal end of the instrument shaft. The return electrode may be located on the instrument shaft, on another instrument or on the external surface of the patient (i.e., a dispersive pad). The proximal end of the instrument(s) will include the appropriate electrical connections for coupling the return electrode(s) and the active electrode(s) to a high frequency power supply, such as an electrosurgical generator.

A more detailed discussion of applications and devices using Coblation® technology as applied to intervertebral discs may be found as follows. Issued U.S. Pat. Nos. 6,283,961; 6,264,651; 6,277,112; 6,322,549; 6,045,532; 6,264,650; 6,464,695; 6,468,274; 6,468,270; 6,602,248; 6,772,012; 7,070,596; and 7,179,255 each of which is incorporated by reference. Pending U.S. application Ser. No. 10/656,597 filed Sep. 5, 2003 which is hereby incorporated by reference.

In one example of a Coblation® device for use with the present invention, the return electrode of the device is typically spaced proximally from the active electrode(s) a suitable distance to avoid electrical shorting between the active and return electrodes in the presence of electrically conductive fluid. In many cases, the distal edge of the exposed surface of the return electrode is spaced about 0.5 mm to 25 mm from the proximal edge of the exposed surface of the active electrode(s), preferably about 1.0 mm to 5.0 mm. Of course, this distance may vary with different voltage ranges, conductive fluids, and depending on the proximity of tissue structures to active and return electrodes. The return electrode will typically have an exposed length in the range of about 1 mm to 20 mm.

A Coblation® treatment device for use in the present invention may use a single active electrode or an array of active electrodes spaced around the distal surface of a catheter or probe. In the latter embodiment, the electrode array usually includes a plurality of independently current-limited and/or power-controlled active electrodes to apply electrical energy selectively to the target tissue while limiting the unwanted application of electrical energy to the surrounding tissue and environment resulting from power dissipation into surrounding electrically conductive fluids, such as blood, normal saline, and the like. The active electrodes may be independently current-limited by isolating the terminals from each other and connecting each terminal to a separate power source that is isolated from the other active electrodes. Alternatively, the active electrodes may be connected to each other at either the proximal or distal ends of the catheter to form a single wire that couples to a power source.

In one configuration, each individual active electrode in the electrode array is electrically insulated from all other active electrodes in the array within the instrument and is connected to a power source which is isolated from each of the other active electrodes in the array or to circuitry which limits or interrupts current flow to the active electrode when low resistivity material (e.g., blood, electrically conductive saline irrigant or electrically conductive gel) causes a lower impedance path between the return electrode and the individual active electrode. The isolated power sources for each individual active electrode may be separate power supply circuits having internal impedance characteristics which limit power to the associated active electrode when a low impedance return path is encountered. By way of example, the isolated power source may be a user selectable constant current source. In this embodiment, lower impedance paths will automatically result in lower resistive heating levels since the heating is proportional to the square of the operating current times the impedance. Alternatively, a single power source may be connected to each of the active electrodes through independently actuatable switches, or by independent current limiting elements, such as inductors, capacitors, resistors and/or combinations thereof. The current limiting elements may be provided in the instrument, connectors, cable, controller, or along the conductive path from the controller to the distal tip of the instrument. Alternatively, the resistance and/or capacitance may occur on the surface of the active electrode(s) due to oxide layers which form selected active electrodes (e.g., titanium or a resistive coating on the surface of metal, such as platinum).

The Coblation® device is not limited to electrically isolated active electrodes, or even to a plurality of active electrodes. For example, the array of active electrodes may be connected to a single lead that extends through the catheter shaft to a power source of high frequency current.

The voltage difference applied between the return electrode(s) and the active electrode(s) will be at high or radio frequency, typically between about 5 kHz and 20 MHz, usually being between about 30 kHz and 2.5 MHz, preferably being between about 50 kHz and 500 kHz, often less than 350 kHz, and often between about 100 kHz and 200 kHz. In some applications, applicant has found that a frequency of about 100 kHz is useful because the tissue impedance is much greater at this frequency. In other applications, such as procedures in or around the heart or head and neck, higher frequencies may be desirable (e.g., 400-600 kHz) to minimize low frequency current flow into the heart or the nerves of the head and neck.

The RMS (root mean square) voltage applied will usually be in the range from about 5 volts to 1000 volts, preferably being in the range from about 10 volts to 500 volts, often between about 150 volts to 400 volts depending on the active electrode size, the operating frequency and the operation mode of the particular procedure or desired effect on the tissue (i.e., contraction, coagulation, cutting or ablation.)

Typically, the peak-to-peak voltage for ablation or cutting with a square wave form will be in the range of 10 volts to 2000 volts and preferably in the range of 100 volts to 1800 volts and more preferably in the range of about 300 volts to 1500 volts, often in the range of about 300 volts to 800 volts peak to peak (again, depending on the electrode size, number of electrons, the operating frequency and the operation mode). Lower peak-to-peak voltages will be used for tissue coagulation, thermal heating of tissue, or collagen contraction and will typically be in the range from 50 to 1500, preferably 100 to 1000 and more preferably 120 to 400 volts peak-to-peak (again, these values are computed using a square wave form). Higher peak-to-peak voltages, e.g., greater than about 800 volts peak-to-peak, may be desirable for ablation of harder material, such as bone, depending on other factors, such as the electrode geometries and the composition of the conductive fluid.

As discussed above, the voltage may be delivered in a series of voltage pulses or alternating current of time varying voltage amplitude with a sufficiently high frequency (e.g., on the order of 5 kHz to 20 MHz) such that the voltage is effectively applied continuously (as compared with, e.g., lasers claiming small depths of necrosis, which are generally pulsed about 10 Hz to 20 Hz). In addition, the duty cycle (i.e., cumulative time in any one-second interval that energy is applied) is on the order of about 50% for the present invention, as compared with pulsed lasers which typically have a duty cycle of about 0.0001%.

The preferred power source of the present invention delivers a high frequency current selectable to generate average power levels ranging from several milliwatts to tens of watts per electrode, depending on the volume of target tissue being treated, and/or the maximum allowed temperature selected for the instrument tip. The power source allows the user to select the voltage level according to the specific requirements of a particular neurosurgery procedure, cardiac surgery, arthroscopic surgery, dermatological procedure, ophthalmic procedures, open surgery or other endoscopic surgery procedure. For cardiac procedures and potentially for neurosurgery, the power source may have an additional filter, for filtering leakage voltages at frequencies below 100 kHz, particularly voltages around 60 kHz. Alternatively, a power source having a higher operating frequency, e.g., 300 kHz to 600 kHz may be used in certain procedures in which stray low frequency currents may be problematic. A description of one suitable power source can be found in commonly assigned U.S. Pat. Nos. 6,142,992 and 6,235,020, the complete disclosure of both patents are incorporated herein by reference for all purposes. In a particular preferred embodiment, the treatment device may be a DC Spinewand®, catalog number K7910-01 and the generator may be a Coblator® Spine System, catalog number KC8000-00, both available from ArthroCare Corporation. The power source may be current limited or otherwise controlled so that undesired heating of the target tissue or surrounding (non-target) tissue does not occur. In a presently preferred embodiment of the present invention, current limiting inductors are placed in series with each independent active electrode, where the inductance of the inductor is in the range of 10 uH to 50,000 uH, depending on the electrical properties of the target tissue, the desired tissue heating rate and the operating frequency. Alternatively, capacitor-inductor (LC) circuit structures may be employed, as described previously in U.S. Pat. No. 5,697,909, the complete disclosure of which is incorporated herein by reference. Additionally, current-limiting resistors may be selected. Preferably, these resistors will have a large positive temperature coefficient of resistance so that, as the current level begins to rise for any individual active electrode in contact with a low resistance medium (e.g., saline irrigant or blood), the resistance of the current limiting resistor increases significantly, thereby minimizing the power delivery from said active electrode into the low resistance medium (e.g., saline irrigant or blood).

The following discussion is an example of the inventive method as applied to a percutaneous intervertebral disc-procedure using Coblation® technology. It is understood that the treatment device of the present invention is not limited to a device utilizing Coblation technology. Instead any other treatment device or treatment modalities (e.g., mechanical instruments, laser, chemical, other RF devices, etc.) may be used in the inventive method either in place of the Coblation® technology or in addition thereto.

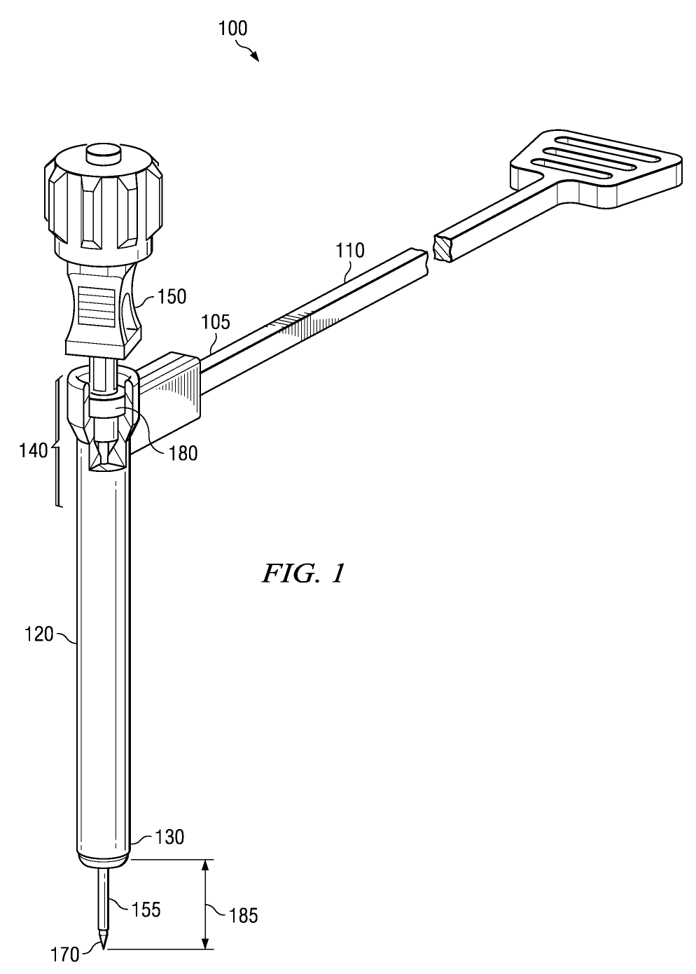

FIG. 1 shows a surgical access system 100 according to the teachings of the present disclosure. System 100 generally includes an access port 105 and a cannula assembly 150. Cannula assembly 150 is sized and configured for slidable insertion and removal from access port 105. Access port 105 includes a port handle 110 and port tubular elongate body 120 connected with port handle 110; port tubular body 120 has a distal end 130 and proximal end portion 140. Body 120 and distal end 130 preferably have a generally atraumatic, smooth and rounded outer surface to minimize any damage to patient tissue.

Cannula assembly 150 generally includes a cannula 155 and a stylet 170 operatively assembled. Cannula assembly 150 may also include a cannula assembly stop 180, removably attached to cannula 155 and formed to nest with proximal end portion 140 of port tubular body 120, to limit extension length 185 of cannula assembly 150 beyond tubular body distal end 130. Once stop 180 is attached, extension length 185 of cannula assembly 150 may be substantially shorter than if stop 180 is removed. Extension length 185 may be selected to provide for differing tissue target locations, alternate access points on a patient, patient size, and/or other variations in patient anatomy. In one embodiment extension length 185 may be 16mm when stop 180 is removed and extension length 185 may be 12 mm once stop 180 is assembled as shown in FIG. 1. In another embodiment (not expressly shown), stop 180 may be placed in any location along cannula 155 to create alternative extension lengths 185.

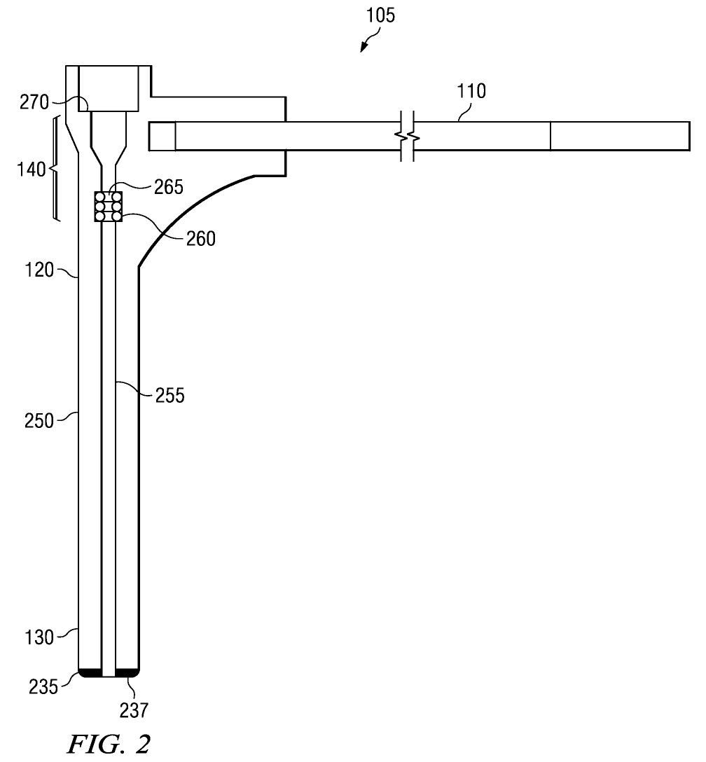

Now referring to FIG. 2, a cross sectional view of access port 105 is shown, including port handle 110 and port tubular elongate body 120. Tubular elongate body 120 has a proximal portion or end 140 and distal portion end 130, an outer surface 250 and inner luminal surface 255. In the present embodiment outer surface 250 is preferably substantially smooth and atraumatic so as to minimize damage to patient tissue during insertion and use. Port handle 110 may be preferably connected with proximal end 140 and facilitates manipulation of access port 105 such that tubular body 120 may be aligned with and provide access to a target tissue. Handle 110 may be a length that allows the user to hold access port 105 in position during imaging but outside the immediate radiographic field.

Access port 105 is preferably constructed from a substantially radiolucent material but also incorporates at least one radiopaque landmark 235 disposed thereon to provide a visual reference when viewed radiographically. In the present embodiment radiopaque landmark 235 is disposed near the access port distal end 130 to provide a visual reference of the alignment of tubular body 110 in relation to a target tissue when viewed radiographically. Radiopaque landmark 235 may be substantially circular but may not be a complete circle in order to function as a reference. In alternate embodiments radiopaque landmark 235 may be one or more dots or lines disposed proximate port distal end 130 and suitable to allow a user to gauge the position of distal end 130 or body 120 when viewed under fluoroscopy or the like. Distal end 130 and distal edge 237 may be substantially smooth so as to minimize tissue damage during use.

Access port 105 may preferably be placed using visual references or using radiographic means, such as a fluoroscope or a CT machine. A surgeon may also use a combination of visual references and radiographic means.

At least one groove 260 may be formed in inner luminal surface 255, sized and shaped to hold one or more frictional component 265. Frictional component 265 is preferably selected so as to create sufficient friction to secure a cannula assembly (as shown in FIG. 1) in place while system is assembled. Groove 260 may be formed substantially around the circumference of inner surface 255 and frictional component 265 may be constructed of an elastomeric material. Frictional component 265 may comprise at least one o-ring. In alternate embodiments, not expressly shown here, frictional components 265 may include at least one individual sphere, at least one tubular component or at least one helical coil, with an appropriately shaped groove or aperture. Frictional component 265 allows a user to release hold of an inserted cannula assembly 150 to perform other tasks (including checking the placement of cannula assembly via a radiographic viewing technology) and said cannula assembly 150 is preferably prevented from sliding freely or otherwise disrupting the placement of cannula 150 with respect to port 105.

In the present embodiment, proximal end portion 140 of tubular elongate body 120 may further comprise a cannula assembly interface 270, formed to interface with a cannula assembly stop, shown in FIG. 1. In the present embodiment interface 270 comprises a nest or nesting surface sized and formed to interface with a cannula assembly stop 180 to selectively limit the extension of cannula assembly 150 beyond tubular distal end 130 of port 105.

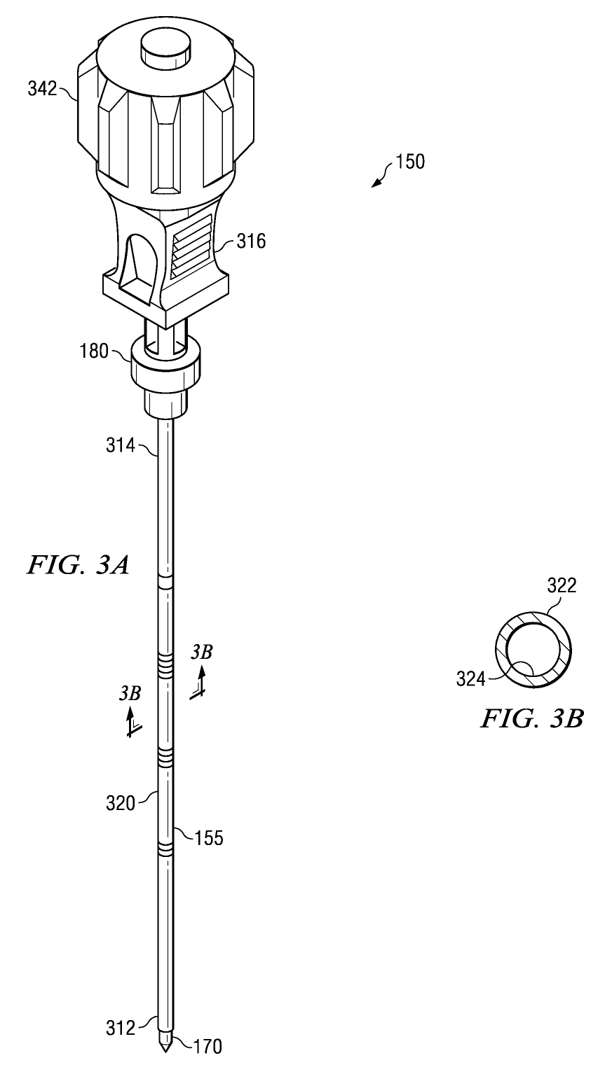

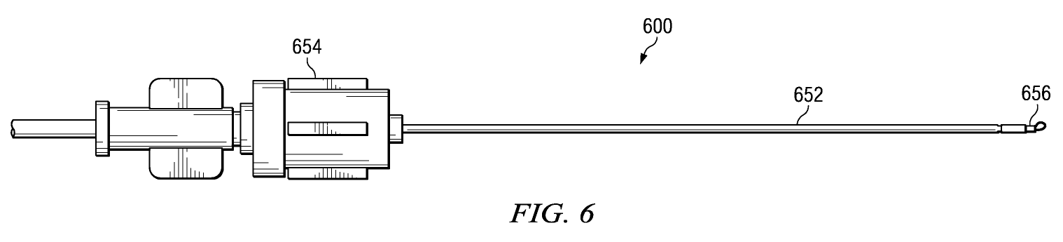

FIG. 3A shows a cannula assembly 150 that generally includes a cannula 155 and a stylet 170 operatively assembled. Stylet 170 is an elongated shaft including a sharp distal terminus, sized and configured for slidable insertion into and removal from cannula 310. Cannula 155 defines a hollow lumen extending from a proximal end 314 to a distal end 312. Distal end 312 may terminate in a tapered or outwardly beveled distal tip 315 for facilitating penetration into bone or tissue surrounding a target site. When assembled, the distal end of stylet 170 extends slightly beyond the distal end 312. A cannula handle 316 is provided at proximal end 314 of cannula 155 which facilitates the user's handling and manipulation of assembly 150. Stylet handle 342 is connected with proximal end of stylet 340. Stylet handle 342 is removably connected with cannula handle 316. Once stylet 170 has gained access to a target tissue, stylet 170 is then removed from cannula 155 and a treatment electrode (as shown in FIG. 6) or other treatment device may then be inserted through cannula 155 and thereby into the target tissue. Cannula handle 316 is adapted for slidable engagement with a treatment electrode (not shown here) after stylet 170 is removed.

Cannula 155 is a tubular elongate body 320 with a substantially smooth outer surface 322 and a generally smooth and preferably inviolate inner luminal surface 324 as shown in FIG. 3B. As shown in FIG. 3A, assembly stop 180 may be removably attached onto outer surface 322 of elongate body 320. As earlier discussed herein, when installed on elongate body 320, assembly stop 180 is generally adapted to interface with a proximal portion 240 of port 105 to limit the extension of cannula 155 from the distal tip 237 of port 105. Stop 180 may be selectively slid along cannula 155 or removed therefrom.

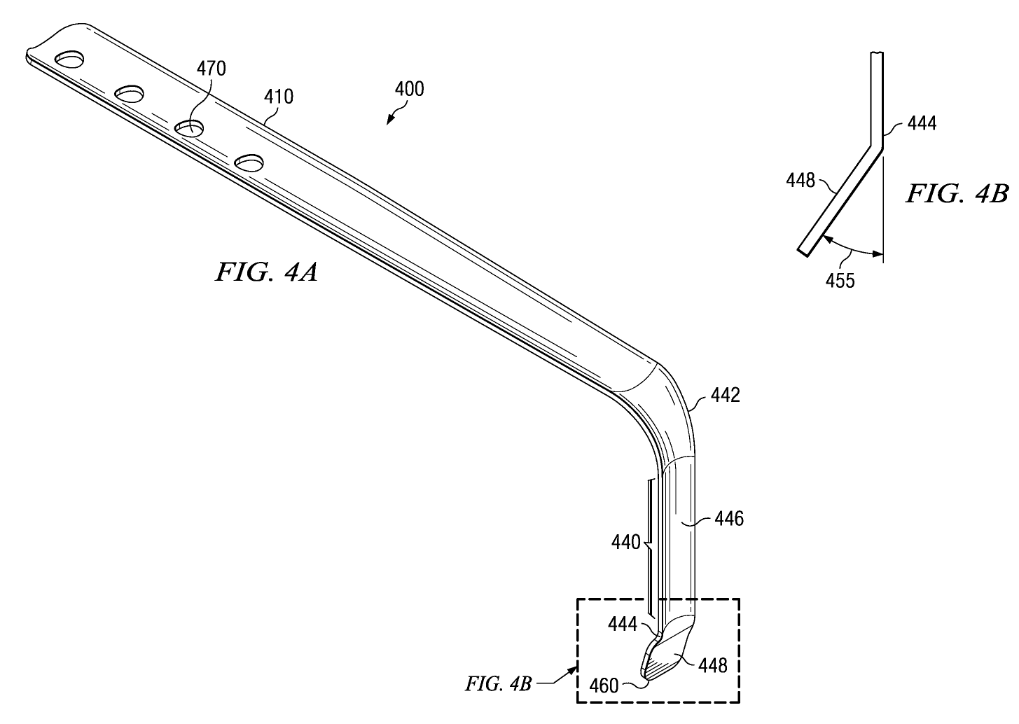

FIG. 4A shows a retractor 400, having a handle 410 and a retraction portion 440 connected to handle 410. Retractor handle 410 may be formed to be long enough to be held outside a radiographic field. Retraction portion 440 is formed to retract body tissue away in order to access target tissue, so as to view the corridor or passageway to the target tissue. Retraction portion 440 has a proximal end 442, a distal end 444 and a curved planar surface 446 there between. Proximate to distal end 444 there may be a substantially flat planar surface 448 formed to provide improved tissue retraction closer to the target tissue. Flat surface 448 may preferably be positioned at an angle 455 relative to curved surface 446, to provide improved tissue retraction closer to the treatment area as shown in FIG. 4B. Retraction portion surfaces 446 and 448 may also have substantially rounded or smooth outer edges 460, so as to reduce unintended tissue damage. However edges 460 may have sufficient edges capable of blunt tissue dissection.

Retractor 400 may be made from substantially lightweight materials and may be radiolucent. Materials may be preferably constructed from biocomposite materials and may include, but are not limited to titanium, titanium alloys, aluminum, aluminum alloys, polymers, glass filled polymers, carbon fibers and resins. Retractor handle 410 may also include voids 470 to reduce the overall weight of retractor 400. Voids 470 may be of any suitable shape and may additionally provide a gripping surface on handle 410 for the user.

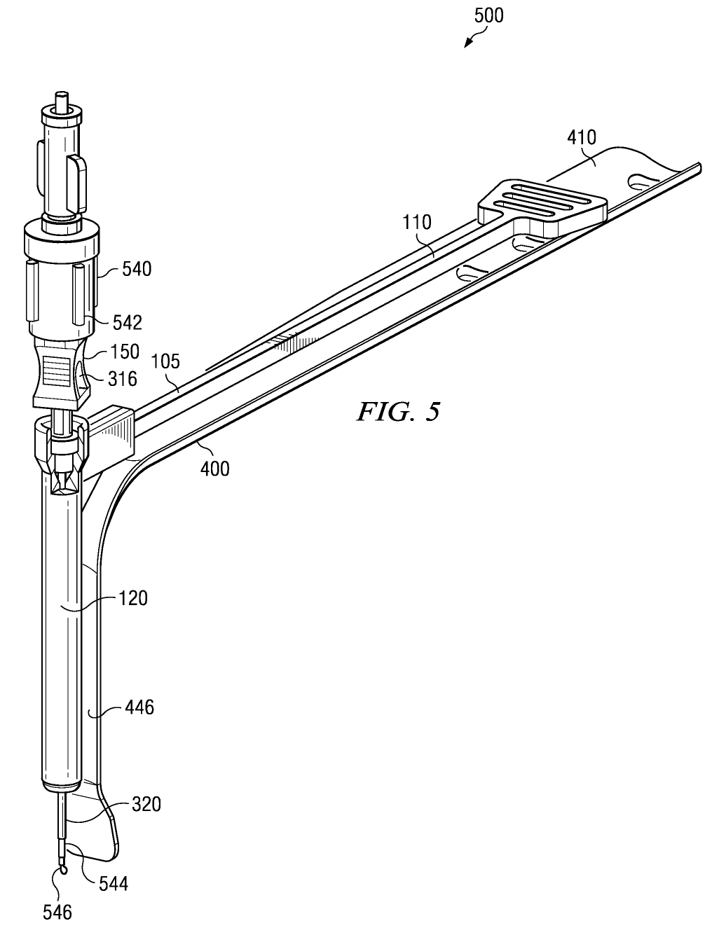

FIG. 5 shows a surgical access assembly 500 including access port 105, retractor 400, and cannula assembly 150 as discussed above and also includes treatment electrode 540. As shown, access port handle 110 and retractor handle 410 may preferably be of substantially complementary shape and size so as to be held by the user together, with one hand. During access to the target tissue however, retractor handle 410 and access port handle 110 may not necessarily be held with the one hand and overlap as shown in FIG. 5. A surgeon may hold retractor handle 410 in one hand and port handle 110 in the other hand, so that retractor handle 410 and port handle 110 are approximately opposite each other. This allows the surgeon to create improved tissue retraction with the retractor 400 while guiding the access port 105 with the second hand. In the present preferred embodiment access port elongate body 120 is formed so as to nest or rest within retraction curved planar surface 446. Also shown on FIG. 5, cannula 150 is inserted into access port tubular body 120. As previously described, cannula 150 generally includes handle 316 and elongated tubular body 320. Handle 316 is adapted to allow removable entry of a stylet (not shown here) or treatment electrode 540. Treatment electrode 540 general comprises a handle 542, elongate body 544 and electrode tip 546.

FIG. 6 illustrates a treatment electrode 600 specifically designed for the treatment of herniated or diseased spinal discs. Referring to FIG. 6, electrode 600 comprises an electrically insulated shaft 652, a handle 654 coupled to proximal end of shaft 652. Handle 654 includes a suitable electrical connector for connecting the treatment electrode with an electrosurgical generator (not expressly shown). Shaft 652 is formed and sized to slidably fit with a cannula (previously described herein). Proximal end of shaft 652 further comprises an electrode tip 656 that may include any suitable configuration of one or more electrodes shapes, designed to treat herniated of diseased spinal discs. In one embodiment, treatment electrode 600 may comprise a DC Spinewand®, catalog number K7910-01 both available from ArthroCare Corporation.

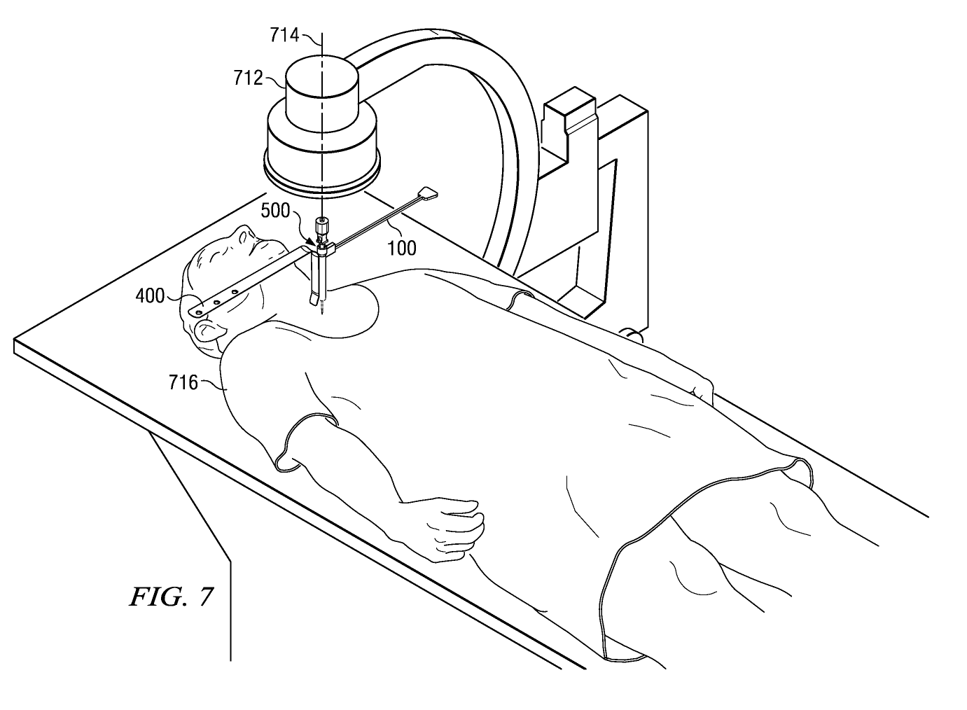

FIG. 7 provides a demonstrative view of how a surgical access assembly 500 may be used in relation to a fluoroscope 712 and patient 716. Surgical access assembly 500 is shown enlarged for clarity. As discussed herein, fluoroscope 712 may refer to any radiographic, fluoroscopic, or other suitable medical imaging apparatus. FIG. 7 shows a patient 716 lying in a supine position with a surgical access assembly 500 inserted with an anterior approach. As described earlier, access assembly 500 includes access system 100 and retractor 400, positioned so that the surgeon may preferably hold system 100 with one hand and retractor 400 with the other. Fluoroscope 712 is in position above said patient 716 and above assembly 500. Fluoroscope 712 has an axis 714 that is shown in alignment with system 718.

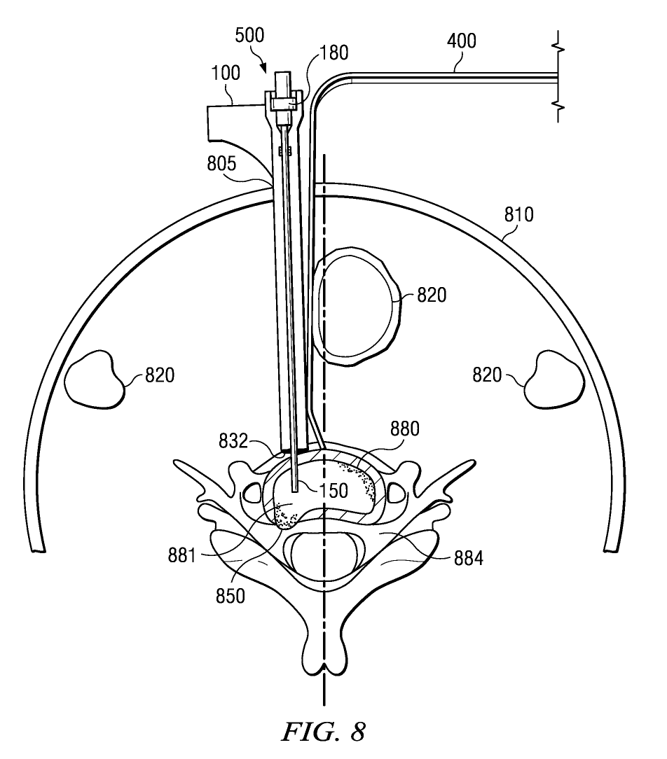

Now referring to FIG. 8 a depiction of a surgical access assembly 500 (with portions removed) is shown entering disc 880 to perform a medical procedure in accordance with teachings of the present disclosure. In the present embodiment disc structure is a cervical disc 880, however in alternate embodiments, surgical access assembly 500 may be used on other intervertebral discs. In this figure stop 180 is shown assembled with access assembly 500. As depicted in the present embodiment access assembly 500 enters the cervical area through skin 810 with an anterior approach 805. The surgeon is careful to avoid any vital structures 820 such as the trachea or jugular veins. Retractor 400 in initially used to create a pathway and vital structures 820 may be retracted. The surgical access system 100 is preferably advanced in the present embodiment until distal end 832 of system 100 abuts intervertebral disc 880. Using a cannula assembly 150, a stylet (not expressly shown here) is further advanced until disc is 880 may be accessed in order to treat contained herniation 850. Stylet is preferably removed and a treatment electrode (not shown here) is inserted to treat and/or remove tissue within the disc. In this figure nucleus pulposus 881 is to be treated so as to reduce nucleus pulposus contained hernia 850 causing impingement on spinal cord 884.

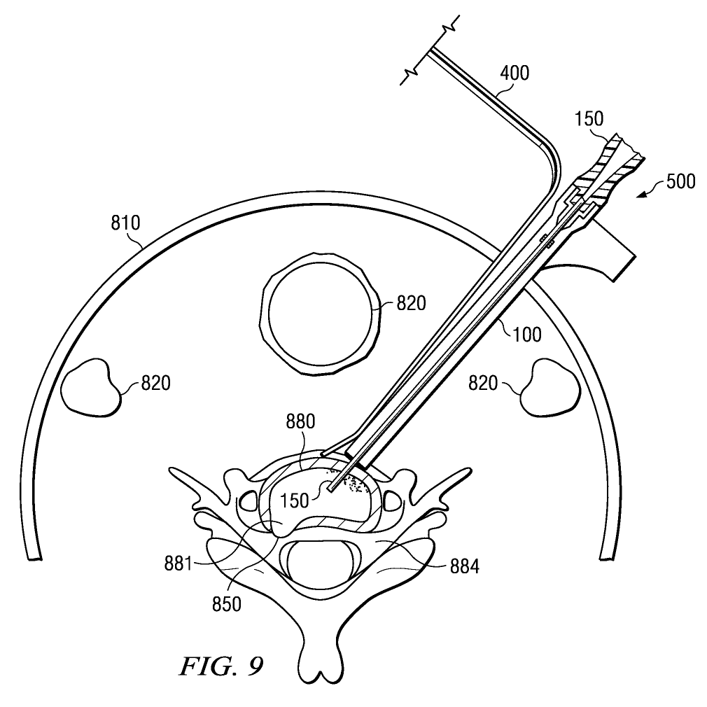

Now referring to FIG. 9 a depiction of surgical access assembly 500 (with portions removed) is shown entering disc 880 to perform a medical procedure in accordance with teachings of the present disclosure. In the present embodiment disc structure is a cervical disc, however in alternate embodiments, surgical device system may be used on other intervertebral discs. As depicted in the present embodiment retractor 400 in first introduced through a minimally invasive opening in skin 810 anterollaterally and then access system 100 is introduced using retractor 400 as a guide. The surgeon is careful to avoid any vital structures 820 such as the trachea or jugular veins. In this figure nucleus pulposus 881 is to be treated so as to reduce nucleus pulposus hernia or swelling 850, causing impingement on spinal cord 884.

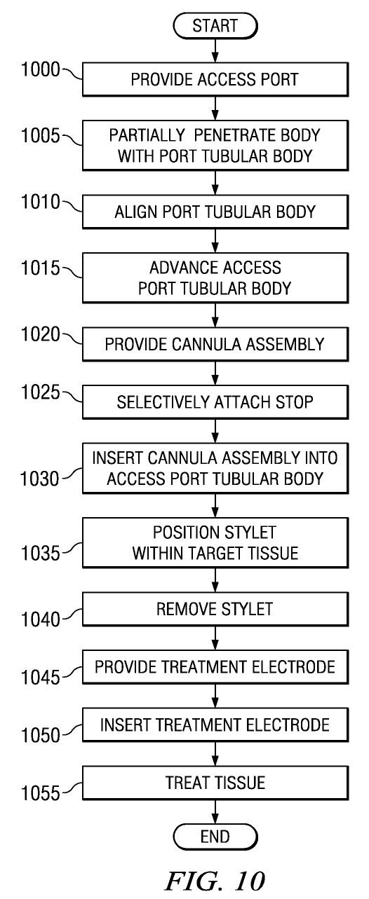

A method of performing a medical procedure using a surgical device system is shown in FIG. 10. An access port is first provided 1000, for accessing target tissue. The tubular body of the access port is then inserted partially into the patient body 1005 towards target tissue. In a preferred embodiment, tubular body is inserted via either an anterior approach (as shown in FIG. 8) or an anterolateral approach (as shown in FIG. 9) to access a cervical intervertebral disc. Using imaging or visual means, the access port elongate body may then be aligned with respect to the target tissue 1010. The access port tubular body is then further advanced towards the target tissue 1015. A cannula assembly comprising an elongate body and outer smooth surface is then provided 1020 and an assembly stop is selectively attached and/or positioned 1025 onto a desired location along the outer surface. The assembly stop may also be selectively removed from the cannula assembly to allow for full penetration with respect to the cannula. The cannula assembly is then inserted 1030 into access port tubular body until the cannula assembly stop (or the handle of the cannula in the event that the stop has been removed) mates with the proximal end of the port tubular body.

The cannula assembly further comprises a stylet, which is then positioned 1035 within the target tissue. The stylet is then removed 1040 from the target tissue and a treatment electrode provided 1045. This treatment electrode is then inserted 1050 into the cannula and the tissue is treated 1055.

Although some embodiments of the present invention have been described, it should be understood that the present invention may be embodied in many other specific forms without departing from the spirit or the scope of the present invention. Therefore, the present examples are to be considered as illustrative and not restrictive, and the invention is not to be limited to the details given herein, but may be modified within the scope of the appended claims.

What is claimed is:

1. A surgical access system for minimally invasive access to a target tissue comprising:
an access port having a port handle and a port tubular elongate body extending from the port handle, the tubular elongate body having a proximal portion and a distal portion; and
a cannula assembly adapted to fit within and be removed from the port tubular body, the cannula assembly having at least one cannula elongate body with a distal end, an outer surface and at least one removable stop, the stop selectively attached to the outer surface of the cannula elongate body and formed to interface with the proximal portion of port tubular elongate body.

2. The system of claim 1 wherein the access port tubular elongate body further comprises an inner luminal surface having at least one frictional component, sized to provide sufficient friction to secure the cannula assembly therewith.

3. The system of claim 2 wherein the frictional device comprises at least one o-ring.

4. The system of claim 2 wherein the inner luminal surface further comprises at least one groove in said inner luminal surface in order to hold said frictional component in place.

5. The system of claim 1 wherein cannula elongate body further comprises a longitudinal axis and the stop is slidably attached to the elongate body outer surface and slidably adjustable along the cannula longitudinal axis.

6. The system of claim 1 further comprising a retractor adapted for retracting tissue within a patient, the retractor having a retractor handle and a retraction portion extending from the retractor handle, the retraction portion having a proximal end, a retractor distal end and an elongated curved surface there between, said curved surface adapted to slidably nest with an outer surface of the access port tubular body.

7. The system of claim 6 wherein the retractor comprises a radiolucent material.

8. The system of claim 6 wherein the distal end of the retraction portion further comprises a substantially flat planar portion for improved retraction.

9. The system of claim 6 wherein the length of the retractor handle is selected to allow the handle to be held outside a radiographic field.

10. The system of claim 6 wherein the distal end of the retraction portion is adapted to blunt dissect soft tissue.

11. The system of claim 6 wherein the port handle and the retractor handle are adapted to be held with one hand.

12. The system of claim 1 wherein the cannula assembly further comprises a stylet adapted to fit within cannula elongate body and adapted to percutaneously access the target tissue.

13. The system of claim 1 further comprising a treatment electrode adapted to fit within cannula elongate body and treat the target tissue.

14. The system of claim 1 wherein the target tissue is cervical disk tissue.

15. The system of claim 1 wherein the access port comprises at least one radiolucent material.

16. The system of claim 1 wherein the distal end of the tubular elongate body comprises at least one radiopaque reference when viewed radiographically.

17. An access port adapted for providing access for a cannula assembly and treatment electrode to a target tissue comprising:
a port handle; and
a port tubular elongate body extending from the port handle, the tubular elongate body having a proximal end and an inner luminal surface, the inner luminal surface further comprising at least one elastomeric frictional component and the proximal end comprising an interface surface, formed to mate with and limit the insertion depth of a cannula assembly.

18. A medical procedure to be performed on a body comprising:
inserting a port tubular body of an access port into a patient towards target tissue, the access port adapted for providing access to a target tissue and having a port handle and a port tubular elongate body extending from the port handle, the tubular elongate body having a proximal end and a distal end;
advancing the access port tubular body further to access the target tissue; and
inserting the cannula assembly into the access port tubular body until the stop mates with the proximal end of the port tubular body, the cannula assembly comprising a cannula, a stylet and a stop adapted to attaching to a desired position on an outer surface of the cannula.

19. The medical procedure of claim 18 further comprising removing the stop from the surface of the cannula to allow for a greater depth of penetration of the cannula when inserted with the access port tubular body.

20. The medical procedure of claim 18 further comprising:
positioning the cannula assembly within the target tissue;
removing the stylet form the cannula assembly;
inserting the treatment electrode into the cannula, the treatment electrode adapted to fit within and be removed from the cannula assembly elongate body; and
treating said target tissue.

21. The medical procedure of claim 20 wherein the target tissue comprises a portion of herniated cervical disk.
